(19) United States
(12) Patent Application Publication
(10) Pub. No.: US 2012/0059488 A1
(43) Pub. Date: Mar. 8, 2012

(54) TRANSPLANTATION DEVICE

(75) Inventor: Shigeto Shimmura, Tokyo (JP)

(73) Assignee: KEIO UNIVERSITY, Minato-ku (JP)

(21) Appl. No.: 13/264,196

(22) PCT Filed: Apr. 15, 2010
(86) PCT No.: PCT/JP10/02751

§ 371 Date: Nov. 16, 2011

Related U.S. Application Data
(60) Provisional application No. 61/264,326, filed on Nov. 25, 2009.

(30) Foreign Application Priority Data
Apr. 16, 2009 (JP) 2009-100267

(51) Int. Cl. A61F 2/02

(52) U.S. Cl. 623/23.72

(57) ABSTRACT

The present invention is intended to provide a transplantation device which can transplant a graft into a layered tissue easily and without damaging the graft or a tissue around the transplantation site. A transplantation device (1) according to the present invention is a device for transplanting a graft to a living body, including a pair of flexible holding sheets (2,3) for holding the graft, wherein each of the holding sheets has a grip part for gripping the holding sheet, and the graft held between the holding sheets is exposed by pulling the paired grip parts (4,5) in directions away from the graft. By using the transplantation device, a graft can be transplanted into a layered tissue easily and without damaging the graft or a tissue around the transplantation site.

### CROSS REFERENCE TO RELATED APPLICATIONS

The present application claims the benefit of the priority to the Japanese Patent Application No. 2009-100267 filed on Apr. 16, 2009 and the U.S. Provisional Patent Application No. 61/264,326 filed on Nov. 25, 2009, disclosures of which are herein incorporated by reference.

### TECHNICAL FIELD

The present invention relates to transplantation devices for transplanting a membranous graft to a living body.

### BACKGROUND ART

The cornea is a transparent tissue with a thickness of about 500 um and located at the outermost layer of an eyeball, which serves as a part of a lens in an ocular refracting system. The cornea consists of the following five layers in the order from outermost: epithelium, Bowman's membrane, corneal stroma, Descemet's membrane and endothelium.

Corneal endothelial cells are known to have very low proliferating and regenerating capabilities in a living body. Therefore, if the endothelial cells are damaged, it is necessary to transplant a sheet of corneal endothelial cells collected from a donor or prepared in culture (Shimmura S. et al., Br. J. Ophthalmol. 89: 134-137, 2005).

The sheet of corneal endothelial cells is transplanted as a graft by incising the cornea of a recipient, inserting the graft to the interior of the cornea, and attaching the graft in the cornea. However, the sheet of endothelial cells tends to curl outwardly and become tubular once it is detached from the multilayered cornea for the purpose of the transplantation. Thus, the transplantation by inserting such a graft to the interior of a cornea of a recipient while it is extended in a membranous form requires sophisticated technique and experience. Currently the transplantation is performed in a method where a graft in a curled form is inserted into a cornea and then extending the graft into a form of a sheet at the transplantation site (Malles G. R. J. et al., Cornea 25 (8): 987-990, 2006), or in a method where a graft in an extended form is placed on a carrier or the like and then inserted into the cornea (Shimmura S. et al., Br. J. Ophthalmol. 89: 134-137, 2005).

However, these methods have some problems such as difficulty in making the sheet of corneal endothelial cells into a fully extended form, as well as damages to the graft and tissues around the transplantation site of the recipient due to excessive handling because of the difficulty.

### SUMMARY OF INVENTION

### Technical Problem

The present invention has an object to provide a transplantation device which can easily transplant a graft into a layered tissue without damaging the graft or a tissue around the transplantation site.

### Solution to Problem

A transplantation device for transplanting a graft to a living body according to the present invention includes a pair of flexible holding sheets for holding the graft, wherein each of the holding sheets includes a grip part for gripping the holding sheet, and the graft held between the holding sheets is exposed by pulling the grip part of at least one of the holding sheets in a direction away from the graft.

In the transplantation device according to the present invention, each of the pair of holding sheets may have a shape and area sufficient to hold the graft without exposing it. The holding sheet may have a thickness of 5 to 100 um. The holding sheet may have a thickness of 50 to 300 um and more preferably the material of the holding sheet is silicone.

In the transplantation device according to the present invention, the holding sheets may be connected to each other at a connecting part, and the grip part may be positioned at a rim of the holding sheet opposite to the connecting part.

The grip part may include a tag.

The transplantation device may be formed of a single seamless sheet.

In the transplantation device, a surface of one of the holding sheets may be coated with a viscoelastic substance, wherein the surface faces the graft. The graft may be membranous, and more preferably the graft is a sheet of corneal endothelial cells.

A method of transplantation according to the present invention includes the steps of placing a graft between a pair of holding sheets, placing the holding sheets at a transplantation site, and exposing the graft at the transplantation site. The above-mentioned transplantation device is preferably used in the method, wherein each of the paired holding sheets has a grip part for gripping the holding sheet, and the graft held between the holding sheets may be exposed by pulling the grip part of at least one of the paired holding sheets in a direction away from the graft. The method of transplantation may further include a step of removing the holding sheet from the transplantation site. For example, the pair of holding sheets may be removed by pulling the grip part of at least one of the paired holding sheets in a direction away from the graft.

### BRIEF DESCRIPTION OF DRAWINGS

FIG. 1 shows a schematic diagram of the transplantation device in one embodiment of the present invention.

FIG. 2 shows schematic diagrams of perforated line (A) and incisions (B) at the boundary between the holding sheets in the transplantation device shown in FIG. 1, as well as schematic diagrams of the exemplary boundary having smaller thickness (C and D), in one embodiment of the transplantation device according to the present invention. C and D represent cross-sectional views of the transplantation device along the line X shown in FIG. 1.

FIG. 3 shows schematic diagrams of a perforated line (A) and incisions (B) at the boundary between the holding sheet and the front tag of the transplantation device shown in FIG. 1, as well as schematic diagrams of the exemplary boundary having smaller thickness (C and D), in one embodiment of the transplantation device according to the present invention. C and D represent cross-sectional views of the transplantation device along the line Y shown in FIG. 1.

FIG. 4 shows a schematic diagram of the transplantation device holding a membranous graft in one embodiment of the present invention.

FIG. 5 shows an insertion of the graft into an eye by using the transplantation device in one embodiment of the present invention.

FIG. 6 shows a photograph of the transplantation device holding the membranous graft in one embodiment of the present invention.

FIG. 7 shows an exposure of the graft by extending the holding sheets of the transplantation device in one embodiment of the present invention.

FIG. 8 shows schematic diagrams of a method for attaching a graft to the interior of a cornea in one embodiment of the present invention.

FIG. 9 shows photographs after the completion of the transplantation of the graft into the cornea in one embodiment of the present invention.

### REFERENCE SIGNS LIST

1: transplantation device; 2: upper holding sheet; 3: lower holding sheet; 4: front tag; 5: rear tag; 6: connecting part.

### DESCRIPTION OF EMBODIMENTS

Hereinafter embodiments of the present invention are described in detail with reference to drawings.

It should be noted that the object, characteristics, advantages and ideas of the present invention will be apparent to those skilled in the art from the descriptions in the present specification, and the present invention can be easily reproduced by a person skilled in the art based on the descriptions in the present specification. The embodiments and specific examples of the invention described herein are to be taken as preferred embodiments of the present invention, and are presented only for illustrative and/or explanatory purposes but not to limit the present invention. It is further apparent to those skilled in the art that various changes and modifications may be made based on the descriptions in the present specification within the intent and scope of the present invention disclosed herein.

==Configuration of the Transplantation Device==

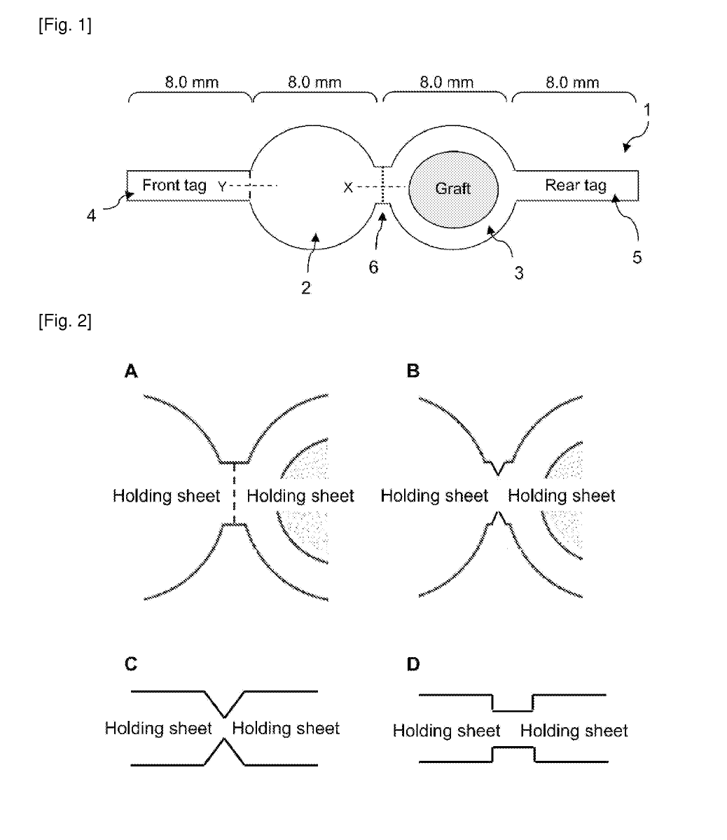

Using the transplantation device according to the present invention, a graft can be transplanted to a living body. The device includes a pair of flexible holding sheets for holding the graft therebetween. Each of the holding sheets includes a grip part for gripping the holding sheet. The transplantation device is configured so that the graft held between the holding sheets can be exposed by pulling the grip part of at least one of the paired holding sheets in a direction away from the graft. FIG. 1 shows an exemplary configuration of the transplantation device in one embodiment of the present invention.

The holding sheet should have rigidity and flexibility appropriate for enabling smooth transplantation of the graft to the graft receiving tissue by using the trans-plantation device. The rigidity and flexibility may depend on the material of the holding sheets or the thickness of the holding sheets composed of the material, but is not particularly limited as long as the sheets can perform the function of holding the graft and inserting the graft into the transplantation site of a recipient.

The material of the holding sheet is not particularly limited and may be appropriately selected by a person skilled in the art as long as it can be formed into a desired shape as a holding sheet and can function as the holding sheet once formed. Preferred examples of the material have bio-nontoxicity, and are exemplified by publicly known materials such as silicone, silicone hydrogel, acryl, fibroin, polyvinyl alcohol, polyhydroxyethyl methacrylate, and the like. The material may also be fibronectin, collagen and the like, having biosolubility. In this case, the transplantation device is not required to be removed after a transplant operation and may be left at the transplantation site. The pair of holding sheets may be composed of different materials, in which case the sheets are preferably connected to each other at a connecting part in view of their operability. The two holding sheets may be connected by an adhesive agent or adhesive means suitable for the material(s). In a preferred embodiment, however, the pair of holding sheets is formed of a single seamless sheet.

The thickness of the holding sheet is not particularly limited as long as the selected material can be actually formed into the thickness and the holding sheet with the thickness can function as the holding sheet once formed. The thickness is preferably equal to or less than 100 um, more preferably 5 to 100 um, and still more preferably 5 to 30 um. In an embodiment where silicone is selected as the material, the thickness is preferably 50 to 300 um, more preferably 100 to 250 um, and most preferably 150 to 200 um.

The surface of the holding sheet may be treated to maintain water-retaining capacity or smoothness, or a treatment for roughing the surface. The treatment may be appropriately selected by a person skilled in the art depending on easiness of handling during the transplantation of the graft, as well as the types of graft receiving tissue and the transplantation site. Furthermore, the surface of the holding sheet may be coated with a viscoelastic substance or the like for protecting the graft or the transplantation site of the recipient, and/or for improving operability of the holding sheet. The viscoelastic substance may be appropriately selected from publicly known materials by a person skilled in the art, and examples include hyaluronic acid and methyl cellulose. A commercially available viscoelastic agent containing one of such viscoelastic substances as an effective ingredient may also be used. In a particularly preferred embodiment, the surface of the holding sheet which faces the graft is coated with the viscoelastic substance, so that smooth transplantation of the graft to the transplantation site can be achieved after the transplantation device is inserted into the transplantation site, as described hereinbelow.

The shape and area of each of the paired holding sheets are not particularly limited and may have a different shape and/or area from each other, but may be appropriately selected by a person skilled in the art as long as the graft held in the holding sheets can be inserted into the transplantation site and the graft is not exposed while being held between the pair of holding sheets. More specifically, the shape may be a diamond, a rectangle or a square with corners, as well as a circle or an ellipse without a corner. The area is preferably larger than that of the graft. In consideration of the steps of inserting the holding sheets with the graft held therebetween to the transplantation site and removing the sheets after the transplantation, the preferred shapes include an ellipse with its major axis aligned with the directions of insertion/removal, and a rectangular or a diamond with its corners being rounded. In a specific embodiment where the transplantation device of the present invention is to be used for a corneal transplantation in an eye, the transplantation device may include holding sheets having the shape of a circle or an ellipse and the area with a diameter of 5 mm to 20 mm.

A three-dimensional shape of the holding sheet may also be appropriately selected by a person skilled in the art depending on the type of graft receiving tissue or the trans-plantation site, and the sheet may be in a flattened shape, or alternatively in a shape with its portion or entirety being lifted like a dome, for example.

The color or clarity of the holding sheet is not particularly limited and may be appropriately selected by a person skilled in the art depending on the type of the graft or its operability at the transplantation site. Preferably the sheet has a color and clarity sufficient to ensure visibility for the transplantation site and its surrounding tissues of the recipient, as well as distinction for the holding sheet by itself, during a transplant operation.

In an embodiment where the pair of holding sheets is to be folded up at a boundary, the boundary may have a cut line such as a perforated line, incisions at its both ends, and/or a thickness smaller than other parts of the holding sheet (FIG. 2). Such a configuration makes it easier to fold the pair of holding sheets at the boundary for holding the graft therebetween, as well as to tear the holding sheets apart at the boundary after transplanting the graft to the graft receiving tissue. In an embodiment where the material of the sheet is silicone and the boundary has a smaller thickness, the boundary may have a thickness of about ¼ to ½ of other parts of the transplantation device. For example, the thickness of the boundary may be, but not limited to, 50 to 100 um when the thickness of other parts of the transplantation device is about 200 um, and may be appropriately select by a person skilled in the art depending on the use of the transplantation device or easiness of handling. When the pair of holding sheets consists of multiple independent holding sheets, the connecting part therebetween preferably forms the boundary.

The position of each of the grip part relative to each of the pair of holding sheets is not particularly limited, but in order for the graft held between the holding sheets to be exposed by pulling the grip part of at least one of the pair of holding sheets in a direction away from the graft, the grip part is preferably positioned at a rim of each of the two holding sheets opposite to the connecting part.

While the grip part of each of the holding sheets may constitute a part of the holding sheet, the grip part is added to the holding sheet in a form as a tag more preferably. In an embodiment where the holding sheet possesses the tag, the tag and the holding sheet may be composed of different materials. More preferably, the tag and the holding sheet do not have a seam therebetween, and the entire transplantation device is composed of a single sheet.

The number of the tag connected to the holding sheet may be one or more, and each of the paired holding sheets may have a different number of the tag(s). The shape of the tag is not particularly limited, as long as the holding sheet can be grasped by gripping the tag(s), and the grip part is connected to the holding sheet so that the graft held between the holding sheets can be exposed by pulling the tag(s) connected to at least one of the paired holding sheets in a direction away from the graft. The tag may be rope-shaped or more preferably strap-shaped in view of its operability. In an embodiment of the transplantation device where one holding sheet includes multiple tags, these tags may be positioned so that the graft held between the holding sheets can be exposed by pulling one of the tags. Alternatively, the tags may be positioned so that a stable force can be applied to the holding sheet for exposing the graft by pulling the multiple tags simultaneously.

The tag may be composed of any of publicly known materials having bio-nontoxicity as long as it can be formed into a desired shape as a tag, and preferred examples include silicone, silicone hydrogel, acryl, fibroin, polyvinyl alcohol, polyhydroxyethyl methacrylate, cotton, polyester, collagen and the like. The material of the tag may be the same as or different from the material of the holding sheet, but in view of the trans-plantation device's operability, the holding sheet and the tag are preferably composed of the same material and formed continuously without a seam therebetween. In an embodiment where the holding sheet and the tag are composed of different materials, they may be connected to each other by any of publicly known connecting means or adhesion means suitable for the materials.

The size of the tag is not particularly limited as long as the tag can function as a grip part of the transplantation device and enable smooth transplantation. In an embodiment where the tag is strap-shaped, it may be 3 to 15 mm long.

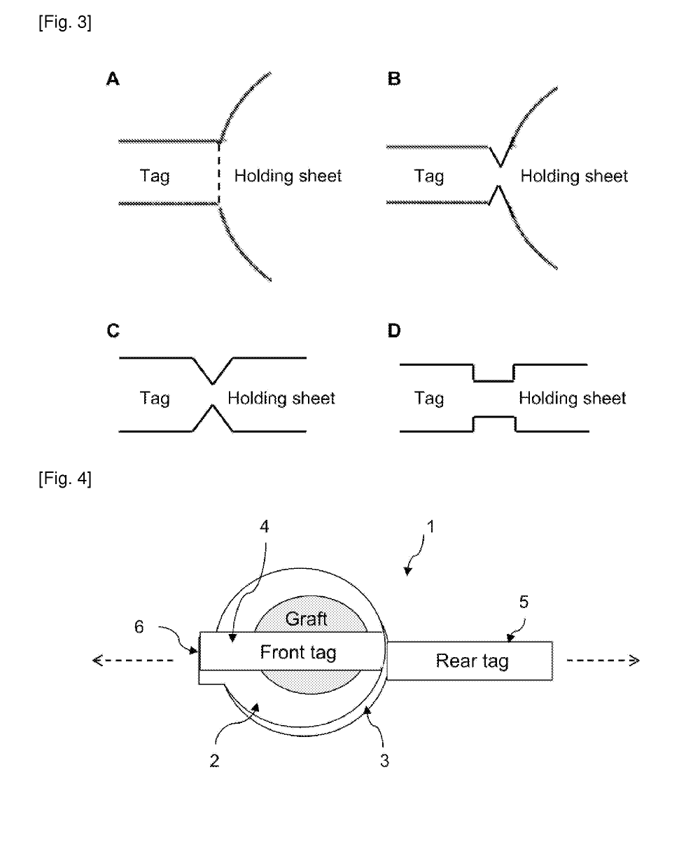

In an embodiment where the tag is strap-shaped and the transplantation device holding the graft is folded up at a boundary between the holding sheet and the tag when the transplantation device holding the graft is inserted into the transplantation site, the boundary may have a cut line such as a perforated line, incisions at its both ends, and/or a thickness smaller than other parts of the holding sheet to enable easy folding (FIG. 3).

In an embodiment where the material of the transplantation device is silicone and the boundary has a smaller thickness than that of other parts of the transplantation device, the boundary may have a thickness of about ¼ to ½ of other parts of the trans-plantation device. For example, the thickness of the boundary may be, but not limited to, 50 to 100 um when the thickness of other parts of the transplantation device is about 200 um, and may be appropriately select by a person skilled in the art depending on the use of the transplantation device or easiness of handling. When the holding sheet and the tag are composed of different materials, the connecting part therebetween preferably forms the boundary.

The boundary between the pair of holding sheets is preferably given smaller strength than the boundary between the tag and the holding sheet, for example, by having a cut line with more perforations, deeper incisions, and/or smaller thickness than the boundary between the tag and the holding sheet. Such a configuration makes it possible to break the boundary between the pair of holding sheets by pulling the paired tags away from each other while keeping the boundary between the tag and the holding sheet connected.

==Method for Manufacturing the Transplantation Device==

The transplantation device of the present invention includes a pair of holding sheets and grip parts. A method for manufacturing the transplantation device is not particularly limited and may be selected from various publicly known methods by a person skilled in the art. For example, the transplantation device can be manufactured by cutting a sheet of material with desired thickness into a shape of the transplantation device. In this case, the entire device may be cut out as one piece of a sheet. Alternatively, each of the holding sheets and the grip parts may be cut out separately and then each part may be connected to form the transplantation device. These methods are suitable for manufacturing the transplantation device from the material that is already in the form of a sheet. In an alternative embodiment, a mold having a desired thickness and shape may be made, and then a material in a liquid form may be injected therein and allowed to set to form the transplantation device. This method is suitable when using a material that can be liquefied depending on conditions, such as silicone or polyvinyl alcohol.

The “silicone” as used herein refers to the materials which can be formed by hardening a polymer essentially composed of organic polysiloxane having the backbone of siloxane bonds between silicon and oxygen. A preferable silicone to be used as a material for manufacturing the holding sheet has the following physical properties: Shore A hardness of 30 to 90, tensile strength of 40 to 100 kg/cm², extensibility of 50 to 500% and tearing strength of 7 to 40 kN/m. The properties other than the hardness can be measured by the assays defined in JIS K6301 standard. The silicone may be prepared by a person skilled in the art appropriately in any of publicly known methods, and may also be purchased as a commercial product from sources such as Momentive Performance Materials, Dow Corning Toray, and Shin-Etsu Chemical. When preparing the silicone, the organic polysiloxane as an essential material is not particularly limited, and may be a commercially available polydimethyl-siloxane, or a polydimethylsiloxane chemically modified by a person skilled in the art in any of publicly known methods. The method for hardening the polymer to prepare the silicone is not particularly limited and may be appropriately selected by a person skilled in the art depending on the properties of the polymer to be used.

==Use of the Transplantation Device==

A graft receiving tissue to which the transplantation device of the present invention may be applied is not particularly limited, and the tissues with which a use of the trans-plantation device would be particularly effective for transplantation include tubular tissues such as blood vessels, tissues having an internal cavity such as heart, intestine and colon, and layered tissues such as cornea and retina of the eye. The “graft receiving tissue” as used herein refers to a tissue of a recipient which receives a graft in transplantation.

The animal species in which a transplantation operation is to be conducted using the transplantation device of the present invention is not particularly limited as long as the animal has any of the abovementioned tubular tissues, the tissues having a cavity and the layered tissues, and preferred examples of the animal are mammals, and more preferred is human.

The shape of the graft is not particularly limited, and the examples of the graft with which a use of the transplantation device would be particularly effective for trans-plantation include a membranous graft and a graft in the form of a sheet.

The source of the graft is not particularly limited, and the graft may be either a piece of tissue collected from a donor, a cultured cell sheet prepared by culturing cells, or an artificially synthesized graft. The donor and the recipient may be the same individual.

In an embodiment where the graft is a piece of tissue collected from a donor, the graft may be derived from any tissue, and is preferably derived from the same tissue as the tissue at the transplantation site. In an embodiment where the graft is a piece of tissue prepared by culturing of cells, the cells may be derived from any source, and the cells are preferably of a cell type present in the tissue at the transplantation site, or of a cell type similar to the cell type present in the tissue at the transplantation site.

In an embodiment where the graft is a synthesized graft, the material of the graft may be, but not limited to, polyester or silicone, and it may be appropriately selected from publicly known materials by a person skilled in the art.

When the transplantation device is used in transplantation, a graft is placed on one of the pair of holding sheets, and the other holding sheet is placed over the graft so that the graft is kept sandwiched between the two holding sheets. Hereinafter the holding sheet which will be removed in the following step of exposing the graft is referred to as the upper holding sheet, whereas the other holding sheet which will remain holding the graft is referred to as the lower holding sheet.

In an embodiment where the transplantation device is formed of a single seamless sheet, the graft may be held between the pair of holding sheets by valley-folding the transplantation device at the connecting part of the paired holding sheets. In an embodiment where the transplantation device includes tags as the grip parts, the seam between the upper holding sheet and the tag connected thereto (also referred to as the front tag) may be set into a mountain fold. The way how the transplantation device and the tag are folded up may be reversed depending on whether the surgeon to conduct the transplantation is left-handed or right-handed.

In order to place the graft on the holding sheet, the graft picked up by forceps etc. may be mounted on the holding sheet. Alternatively, the holding sheet may be laid on top of the extended graft and then the holding sheet may be picked up from its one end while keeping the graft attached by surface tension. In an alternative embodiment where the graft is a piece of tissue, the graft may be prepared by cell culture or tissue culture over the holding sheet.

The graft is held between the holding sheets so that a side of the graft that will contact with the graft receiving tissue faces the upper holding sheet. In an embodiment where the graft is a piece of tissue or a cultured cell sheet, the graft would gain directionality due to polarity of the cells (In the graft as a piece of tissue or a cultured cell sheet, the side which will contact with a tissue of a transplantation site or a basal membrane is hereinafter referred to as the basal side, and the other side is referred to as the apical side.). In such an embodiment, the graft may be held so that the basal side faces the upper holding sheet and the apical side faces the lower holding sheet.

Prior to the transplantation, the transplantation device holding the graft is inserted into the transplantation site of a recipient, i.e., to the interior of a tubular tissue or a tissue with cavity, or to the space between layers of a layered tissue. The method for the insertion is not particularly limited as long as the entire transplantation device can be inserted into the transplantation site without exposing the graft during the inserting step. The transplantation device may be rolled up while holding the graft and then inserted into the transplantation site by using an injector or the like. Alternatively, an end of the transplantation device holding the graft may be gripped by forceps etc. and pulled into the transplantation site. In this case, the transplantation device holding the graft is inserted into the transplantation site so that the upper holding sheet of the trans-plantation device will face the graft receiving tissue of the recipient.

Then, each of the grip parts of the two holding sheets is gripped by forceps etc., and at least one of the grip parts is moved by pulling in a direction away from the graft, and the upper holding sheet is removed from the graft so that the graft placed between the holding sheets become exposed. For example, if the upper holding sheet is moved for removal, the graft will remain on the lower holding sheet. On the other hand, if the lower holding sheet is moved, the graft remains held by and moves together with the lower holding sheet, resulting in the removal of the upper holding sheet and the exposure of the graft. In these cases, the other holding sheet may be lightly pulled in an opposite direction so that it would not be moved. Alternatively, both the holding sheets may be pulled simultaneously in opposite directions so that both the holding sheets are moved to expose the graft.

As mentioned above, a coating of the surface of the lower holding sheet, which faces one side of the graft, with a viscoelastic substance etc. will ensure the graft to remain on the lower holding sheet when the other side of the graft is exposed, thereby making it easy to remove the upper holding sheet while keeping the graft extended.

After the one side of the graft is exposed, the lower holding sheet may be optionally moved to adjust the position of the graft being held on the lower holding sheet relative to the graft receiving tissue. Then the exposed side of the graft may be pressed against the graft receiving tissue. The pressing of the graft against the graft receiving tissue may be made by giving a force to the lower holding sheet on its side opposite to the side facing the graft, thereby achieving an effective adhesion of the graft to the graft receiving tissue. In one embodiment, the graft may be pressed by injecting air or a viscoelastic substance to the side of the lower holding sheet opposite to the side facing the graft. Alternatively, a biological adhesive such as fibrin glue may be injected into the space between the graft and the graft receiving tissue to adhere the graft to the graft receiving tissue.

After the graft is thus transplanted to the graft receiving tissue of the recipient, the lower holding sheet may be removed from the transplantation site. The lower holding sheet can be pulled out in one direction by gripping the grip part of the lower holding sheet by forceps etc. and pulling the transplantation device in a direction away from the transplantation site. In an embodiment where the pair of holding sheets is formed of a single sheet, a grip part of either one of the paired holding sheets, may be pulled in a direction away from the transplantation site. Alternatively, the grip parts of both the holding sheets may be gripped by forceps etc. respectively and pulled away from each other in opposite directions so that a part of the sheet is torn apart and the trans-plantation device thus separated is pulled out in two directions. It is noted that in an embodiment where the transplantation device is composed of a biosoluble material, the device may be left at the transplantation site.

==Exemplary Use of the Transplantation Device==

A method for using the transplantation device will be hereinafter explained with reference to an exemplary transplantation device, as shown in FIG. 1, which has two holding sheets in the same circular shape and paired tags as the grip parts, wherein the holding sheets and the tags are formed of a single seamless sheet.

When using the transplantation device for a transplantation, as shown in FIGS. 1 and 2, a graft is placed on the lower holding sheet 3, that is one of the paired holding sheets, a connecting part 5 of the transplantation device is valley folded, and the other of the holding sheets as the upper holding sheet 2 is laid on top of the graft, thereby holding the graft between the pair of holding sheets 2 and 3. Then, a seam between one of the tags 4 (also referred to as the front tag) and the upper holding sheet 2 is mountain folded. It is preferable to coat a surface of the lower holding sheet 3 which faces the graft with a viscoelastic substance etc. before use of the transplantation device.

The transplantation device thus holding the graft is inserted to a transplantation site of a recipient. Then, each tip of the front tag 4 and a rear tag 5 is gripped by forceps etc., and the front tag 4 is pulled in a direction away from the graft (FIG. 4, dashed arrow). The tag 5 may be lightly pulled at this time in an opposite direction away from the graft, so that the lower holding sheet 3 will not move. The upper holding sheet 2 and the lower holding sheet 3 are thus expanded to expose one side of the graft held inside, and the lower holding sheet 3 is pressed against the graft receiving tissue.

Afterwards, either of the tag 4 or 5 of respective holding sheets 2 and 3 is gripped by forceps etc., and the transplantation device is pulled in a direction away from the trans-plantation site to remove the transplantation device in one direction. Alternatively, both the tags 4 and 5 may be gripped respectively by forceps etc. and pulled away from each other in opposite directions so that the transplantation device is torn apart at the connecting part 6 and the holding sheets thus separated are removed in two directions.

### EXAMPLES

In this example, a transplantation device according to the present invention is used for a transplantation of a sheet of corneal endothelial cells in the eye.

==Recipient==

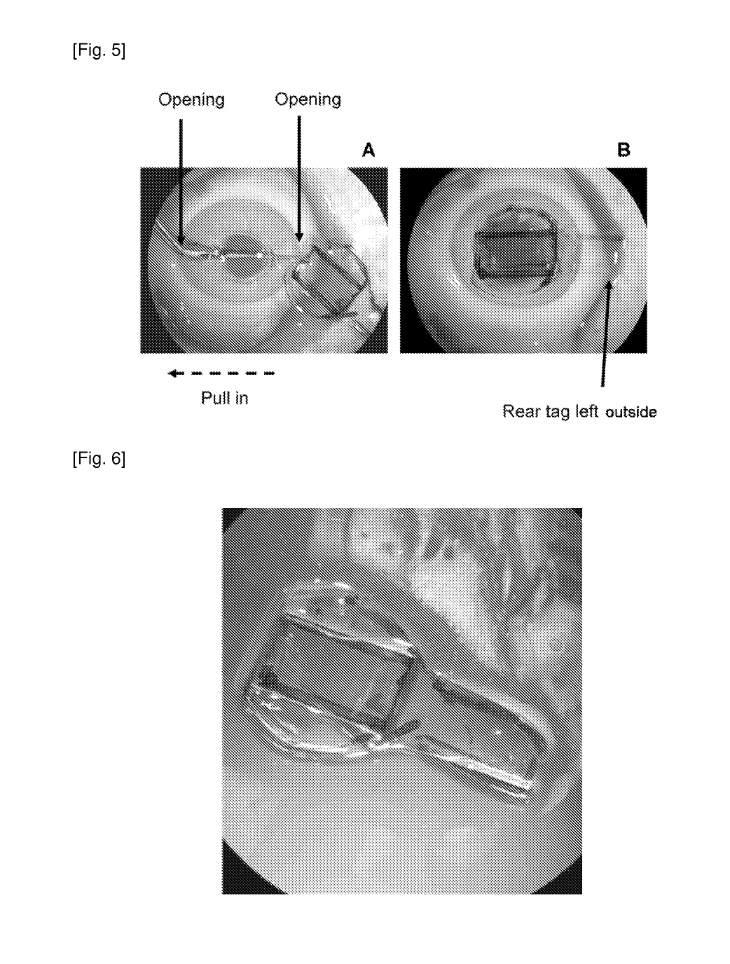

Each of four domestic rabbits (female, 3 kg, Shiraishi experimental animal breeding farm) was anesthetized by intramuscular injection of 4 ml ketamine/xylazine mixed anesthetic (1:7) and then local administration of xylocalne. Tropicamide was further administered locally to induce mydriasis, and the anterior chamber of the eye was filled with heparin (5 U/ml). 5 mm openings were made at two opposing positions of the corneoscleral rim down to the plane of Schlemm's canal (FIG. 5A), and Descemet's membrane was hydrodissected by injecting 0.04% trypan blue into the supra Descemet space using a 27 G needle. Following injection of air into the anterior chamber, a circular section of the Descemet's membrane was removed by the CCD method using a 27 G needle.

==Donor==

Each of two domestic rabbits (female, 3 kg) was administered with lethal dose of pentobarbital. A 3 mm incision was made at the corneoscleral rim, and the Descemet's membrane was dissected by an injection of 6.25 mg/ml of indocyanine green so that the Descemet's membrane of the donor could be stained in green color by the indocyanine green. The sclera and cornea were detached and a section of Descemet's membrane with a diameter of 6 mm was made by using a trephine. The endothelium of the Descemet's membrane thus obtained was protected by a viscoelastic substance (hyaluronic acid) and used as a graft.

==Transplantation Device==

The transplantation device to be used included a pair of holding sheets (2, 3) with diameters of 8 mm and a pair of tags (4, 5) with lengths of 8 mm as shown in FIG. 1. These parts were formed of a single seamless sheet of fibroin with a thickness of 100 um. A connecting part (6) between the two holding sheets included a perforated line. A surface of the holding sheet (3) which would face the graft was coated with the viscoelastic substance.

==Transplantation==

The graft from the donor prepared as above was picked up by forceps, mounted on the holding sheet (3) of the transplantation device, and extended by the forceps. As shown in FIG. 4 and FIG. 6, the connecting part (5) of the transplantation device was valley folded, and a seam between the front tag (4) and the upper holding sheet (2) was mountain folded.

A pair of forceps was inserted into the cornea of the recipient through one of the openings on the cornea, and the pair of holding sheets (2, 3) and the front tag (4) of the transplantation device were grasped all together by the forceps and pulled in (FIG. 5A) so that the transplantation device was inserted into the cornea (FIG. 5B). The rear tag (5) was left protruding out of the cornea.

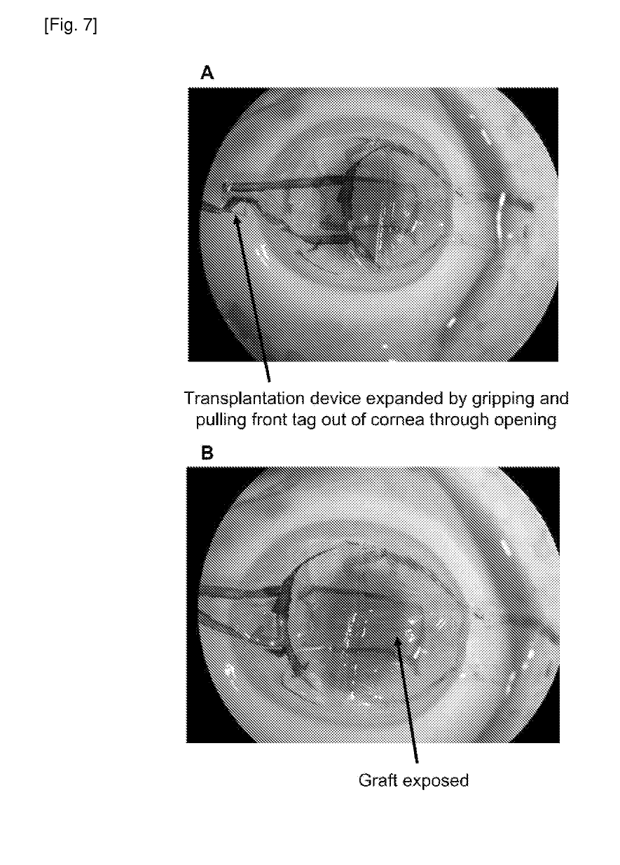

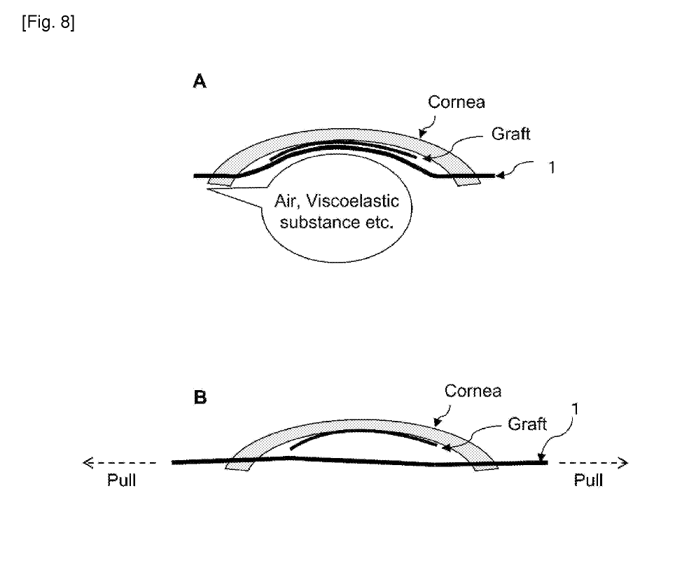

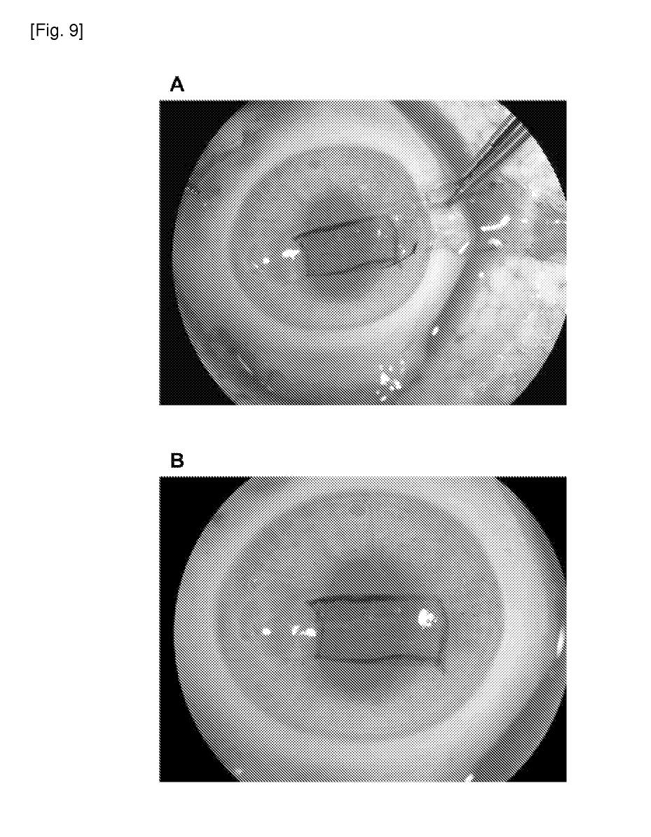

The front tag (4) of the transplantation device was then gripped by forceps and pulled in a direction away from the cornea through the other opening opposite to the one used for pulling the transplantation device into the cornea, so that the transplantation device was expanded (FIG. 7A) and the graft was exposed (FIG. 7B). Air was injected into a space between the surface of the lower holding sheet (3) which was not facing the graft and the endothelium (FIG. 8A), and the graft was allowed to attach to the corneal stroma where the graft was to be received (FIG. 8). Subsequently, the front tag (4) and the rear tag (5) extruding from the cornea through respective openings were gripped respectively by forceps and pulled in opposite directions away from the graft receiving site to detach the graft from the lower holding sheet (FIG. 8B). The front tag (4) and the rear tag (5) were further pulled in opposite directions to tear apart the holding sheets at the perforated line in the connecting part (6) between the upper holding sheet (2) and the lower holding sheet (3), which were then pulled out of the cornea through each of the openings (FIG. 9A), thereby leaving only the graft within the cornea (FIG. 9B).

As described so far, by using the transplantation device of the present invention, a graft could be transplanted into a transplantation site smoothly without excessive handling. In this way, a membranous graft that would otherwise curl can be easily transplanted while kept extended flatly.

### INDUSTRIAL APPLICABILITY

The transplantation device according to the present invention can be used to transplant a graft into a layered tissue easily and without damaging the graft or a tissue around the transplantation site.

1. A transplantation device for transplanting a graft to a living body, comprising
a pair of flexible holding sheets for holding the graft therebetween, wherein
each of the holding sheets comprises a grip part for gripping the holding sheet, and
the graft held between the holding sheets is exposed by pulling at least one of the paired grip parts in a direction away from the graft.

2. The transplantation device according to claim 1, wherein
each of the pair of holding sheets has a shape and area sufficient for holding the graft without exposing the same.

3. The transplantation device according to claim 1 or 2, wherein
the holding sheet has a thickness of 5 to 100 um.

4. The transplantation device according to claim 1 or 2, wherein
the holding sheet has a thickness of 50 to 300 um, and the material of the holding sheet is silicone.

5. The transplantation device according to any one of claims 1 to 4, wherein the holding sheets are connected to each other at a connecting part, and the grip part is positioned at a rim of the holding sheet opposite to the connecting part.

6. The transplantation device according to any one of claims 1 to 5, wherein
the grip part comprises a tag.

7. The transplantation device according to any one of claims 1 to 6, wherein
the transplantation device is formed of a single seamless sheet.

8. The transplantation device according to any one of claims 1 to 7, wherein
a surface of one of the pair of holding sheets is coated with a viscoelastic substance, wherein the surface faces the graft.

9. The transplantation device according to any one of claims 1 to 8, wherein
the graft is membranous.

10. The transplantation device according to claim 9, wherein
the membranous graft is a sheet of corneal endothelial cells.
